(19) United States
(12) Patent Application Publication
(10) Pub. No.: US 2011/0071427 A1
(43) Pub. Date: Mar. 24, 2011

(54) CRYOSURGICAL INSTRUMENT FOR OBTAINING A TISSUE SAMPLE, METHOD FOR CHILLING A PROBE HEAD OF A CRYOSURGICAL PROBE

(76) Inventors: Klaus Fischer, Nagold (DE); Daniel Schäller, Tubingen (DE); Matthias Voigtländer, Gomaringen (DE); Mara Szyrach, Tubingen (DE); Irina Sigle, Mossingen (DE); Lars Blobel, Ammerbuch-Entringen (DE); Markus Enderle, Tubingen (DE)

(21) Appl. No.: 12/994,003

(22) PCT Filed: Apr. 23, 2009
(86) PCT No.: PCT/EP2009/002971

§ 371 Date: Dec. 3, 2010

(30) Foreign Application Priority Data
May 23, 2008 (DE) 10 2008 024 946.7

(51) Int. Cl. A61B 10/02; A61B 10/04; A61B 18/02

(52) U.S. Cl. 600/565; 606/22

(57) ABSTRACT

A cryosurgical instrument for obtaining a tissue sample with a probe, including the probe including a probe head onto which the tissue sample is frozen and a probe shank for guiding the probe head to the tissue sample, a supply means for supplying a fluid into an expansion chamber configured such that supplied fluid expands therein for cooling the probe head, and a removal means connected to the expansion chamber for removing the expanded fluid therefrom via at least one opening on a distal end of the probe to remove the expanded fluid to an outer region outside the probe. A method for cooling a probe head of a cryosurgical probe including supplying a fluid to an expansion chamber near the probe head, expanding the fluid so that heat energy is withdrawn from the probe head, and removing the expanded fluid in a distal region of the probe.

### FIELD OF THE DISCLOSED EMBODIMENTS

The disclosed embodiments relate to a cryosurgical instrument for obtaining a tissue sample and to a method for cooling a probe head of a cryosurgical probe.

### BACKGROUND

In cryosurgery, the taking of tissue samples (biopsy) is facilitated, inter alia, by the targeted use of cold. In this case, a cryoprobe is brought close to a specific tissue region via an endoscope. While the probe head contacts the tissue to be removed, the probe head is cooled in such a way that at least certain portions of the tissue freeze solid on the probe head. After the surrounding tissue has been severed, the sample (biopsate) can be retracted into the endoscope and made accessible for tests.

The cooling power can be provided, for example, by way of targeted use of the Joule-Thomson effect. In this case, a fluid, in particular a gas, experiences a change in temperature as a result of restriction (change in pressure). In cryosurgery, gas is expanded under high pressure into an expansion chamber in such a way that the volume thereof increases in size. In this case, the average particle spacing of the fluid increases, causing its temperature to fall.

A corresponding cryoprobe is known from U.S. Pat. No. 7,156,840 B2. Cryoprobes of this type may also be used in thin hollow organs, such as for example bile or pancreatic ducts, in order to carry out histology of the tissue.

An important criterion for the biopsy is the quality of the sample taken. Removal of the sample should not lead to any mechanical deformation of the sample.

Flexible micro-endoscopes are used to work in the bile and pancreatic duct under endoscopic view. The outer diameter of this flexible micro-endoscope is very small, for example ≦3 mm. Only instruments having a much smaller outer diameter can be introduced into the working channel of endoscopes of this type. Frequently, the outer diameter of the instruments used here may not exceed one millimeter.

Furthermore, in this field of use, the distances between the inlet opening of the endoscope and the working region are very long. The working channel of the endoscope is also correspondingly long. The instrument introduced into the endoscope, for example the cryosurgical instrument with the cryoprobe, must therefore have corresponding dimensions. For example, a length of 180 cm may be required.

A further requirement for the use of a cryoprobe in the above-described specialist field is the provision of sufficient cooling power, so that the biopsate can be fixed sufficiently securely to the tip of the probe. The cooling power is decisively determined by the pressure differential in the expansion chamber. That is to say, the higher the differential is between the pressure of the gas prior to issuing via a nozzle opening and the pressure within the expansion chamber, the higher the cooling power is too. The smaller the corresponding gas feed line is, the more difficult it is to provide a sufficiently high pressure before the nozzle opening. Furthermore, the outflowing gas must be removed from the expansion chamber in order to maintain the pressure gradient. Gas returns having a sufficiently large diameter are conventionally provided for this purpose. However, with the smaller diameter of the return lines, the flow resistance, and thus the pressure within the expansion chamber, rises.

For these reasons, it has proven extremely difficult to provide cryoprobes which have the required dimensions and at the same time provide sufficient cooling power. Conventional biopsy methods are therefore frequently used. Such methods include, for example, the obtaining of samples by means of a brush, or needle biopsy.

However, the quality of the samples thereby obtained is conventionally much lower than that obtainable by a cryobiopsy.

### SUMMARY

The disclosed embodiments provide an improved cryosurgical instrument. In particular, disclosed embodiments include a cryosurgical instrument for obtaining a tissue sample that is suitable for use in thin hollow organs. Furthermore, disclosed embodiments also include a corresponding method for cooling a probe head of a cryosurgical probe.

In the cryosurgical instrument for obtaining a tissue sample with a probe of the disclosed embodiments, the probe includes a probe head for freezing-on tissue, a probe shank for guiding the probe head up to the tissue, a supply means for supplying a fluid, in particular a gas, into an expansion chamber, and a removal means which is connected to the expansion chamber for removing the fluid, the expansion chamber being embodied in such a way that the supplied fluid expands for cooling the probe head.

The cryosurgical instrument according to the disclosed embodiment is distinguished from those previously known in that the removal means includes on the distal region of the probe at least one opening which removes the fluid into an outer region outside the probe.

In other words, the fluid is returned only partially (or not at all) within the probe. The gas supplied via the supply means spreads out in the expansion chamber and is removed from there into an outer region outside the probe. Thus, the provision of a return line within the probe may be partly or wholly dispensed with. The supply means can be made accordingly larger in order to provide sufficient pressure in the expansion chamber. Frequently, the issuing amounts of fluid are so small that they do not cause any damage within the organs. Nor does the fluid used present any risk to the patient. If the fluid is nevertheless to be removed, a separate mechanism may be provided for this purpose. The working channel of the endoscope used may be used, for example, for removing the fluid.

In the probe according to the disclosed embodiments, the probe body defines an outer region and an inner region. Obviously, the fluid is removed out of the inner region into the outer region in conventional probes too. However, the corresponding means are located not on the distal region of the probe, but in the proximal region thereof.

The cryosurgical instrument can include at least one opening at the distal end of the probe shank and/or in the probe head. That is to say, the distal region is defined in such a way as to include the probe head and the distal end of the probe shank.

The cryosurgical instrument can include an adapter for removing the fluid in the proximal direction. An adapter of this type can for example introduce the fluid issuing from the probe into the working channel of an endoscope. Furthermore, the adapter can be configured so as to provide a removal means for the fluid. This removal means guides, like conventional cryosurgical instruments, the fluid to the proximal end of the probe.

The adapter can include a protective tube with a seal, the probe being movably mounted in the protective tube and the seal sealing from the probe an interior or inner region of the protective tube for receiving the fluid. The fluid is therefore introduced via the supply means into the expansion chamber, passes from there into the outer region of the probe via at least one opening, the probe being surrounded by the protective tube in such a way that an intermediate space is produced between the probe and protective tube. The fluid can be guided into this intermediate space in the proximal direction. The seal at the distal end of the protective tube ensures that the fluid cannot escape in the distal direction.

The protective tube can include a region for receiving the tissue sample. According to the disclosed embodiments, the tissue sample, which is fastened to the probe head, can therefore be retracted in such a way that it comes to lie within the protective tube. This allows the tissue sample to be protected from external influences, in particular mechanical loads.

Generally, the protective tube can serve to convert the probe according to the disclosed embodiments so as to allow it to be used also in regions in which it is undesirable for fluid to issue close to the region of the operation.

As previously described, the adapter can be adapted for introducing the fluid into a working channel of an endoscope.

The at least one opening on the distal region of the probe can be arranged laterally of the probe shank or form the probe head in the form of a particle filter. The particle filter can for example have openings having a diameter of approx. 4 μm.

Disclosed embodiments also include a method for cooling a probe head on the cryosurgical probe. The method includes the steps of supplying a fluid, in particular a gas, expanding the fluid in such a way that heat energy is withdrawn from the probe head, and removing the expanded fluid. The step of removing the expanded fluid includes diverting the fluid from the interior of the probe in the distal region of the probe.

In this case too, a basic idea of the disclosed embodiments is that, in the cooling method, the fluid is removed in the distal region and, as a result, separate returning of the fluid is not necessary. In the method, the fluid can be supplied over a period of time which is ≦5 seconds. As a result, only very small amounts of gas are released that enter the outer region of the probe.

### BRIEF DESCRIPTION OF THE DRAWINGS

The disclosed embodiments will be described in greater detail, pointing out further features and advantages, by reference to the example embodiments illustrated in the drawings.

FIG. 1 is a schematic view of a cryosurgical instrument.

FIG. 2 is a schematic view of the cryosurgical instrument from FIG. 1 within an endoscope in clinical use.

FIG. 3 is a schematic view of the distal end of a rigid cryoprobe according to the prior art.

FIG. 4 is a schematic view of the distal end of a flexible cryoprobe according to the prior art.

FIG. 5 is a schematic view of the distal end of a cryoprobe according to one disclosed embodiment.

FIG. 6 is a schematic view of the distal end of a flexible cryoprobe according to the prior art.

FIG. 7 is a schematic view of the distal end of a cryoprobe according to another disclosed embodiment.

FIG. 8 is a schematic view of the distal end of a cryoprobe according to another disclosed embodiment, with a protective tube.

FIG. 9 shows a cryoprobe according to disclosed embodiments in the working channel of a flexible endoscope.

FIG. 10 shows a cryoprobe according to disclosed embodiments with a protective tube and biopsate.

FIG. 11 shows the cryoprobe of FIG. 10 with the biopsate retracted.

FIG. 12 is a schematic view of the distal end of another disclosed embodiment of a cryoprobe.

### DETAILED DESCRIPTION

The same reference numerals will be used in the following description for identical and equivalent parts.

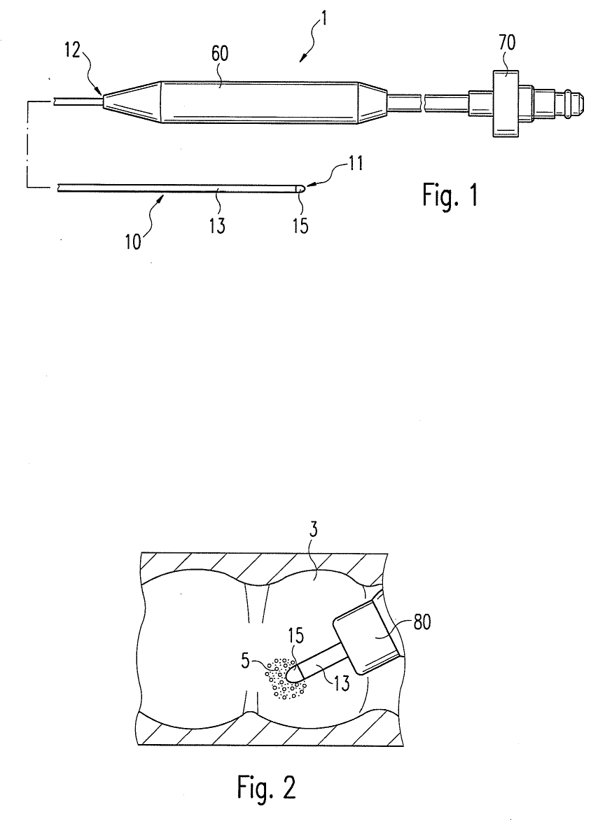

FIG. 1 shows schematically the construction of a cryosurgical instrument 1. The cryosurgical instrument includes a handle 60 for guiding the cryoprobe 10, the cryoprobe 10 being composed of a shank 13 and a probe head 15. The probe head 15 forms the distal end 11 of the cryoprobe 10. The proximal end 12 of the cryoprobe 10 directly adjoins the handle 60. A connection 70, which can be used to connect the cryosurgical instrument 1 to a compressed air source, is located at the proximal end of the handle 60. The compressed air source supplies the cryosurgical instrument 1 with compressed air.

FIG. 2 shows the cryoprobe 10 from FIG. 1 in clinical use. The shank 13 of the cryoprobe 10 is located within the working channel of an endoscope 80, the endoscope 80 being introduced into a hollow organ, for example a portion of the gut. The shank 13 and also the probe head 15 protrude beyond the distal end of the endoscope 80 and contact a region of the tissue 3 of the gut. The probe head 15 can be cooled by means of the Joule-Thomson effect in such a way that the region freezes solid on the probe head 15. A tissue sample 5 may be separated out from the tissue 3 by way of a mechanical pull.

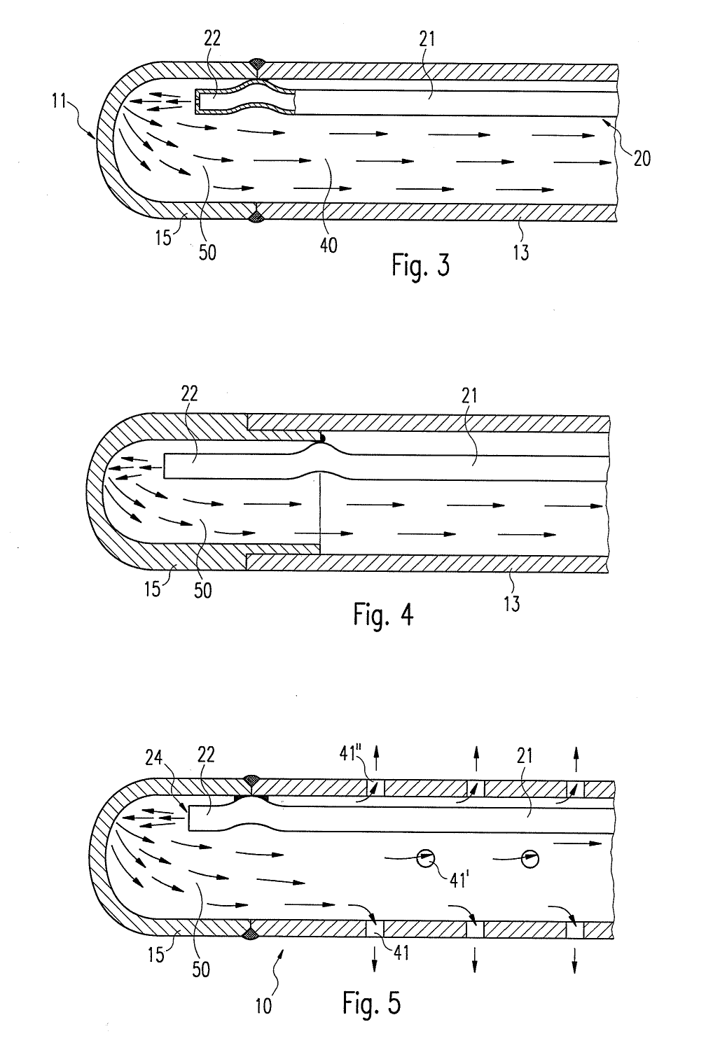

FIGS. 3 and 4 are cross-sections of the distal end 11 of a cryoprobe 10, according to the prior art. A distinction is conventionally drawn between rigid cryoprobes 10 (FIG. 3) and flexible cryoprobes 10 (FIG. 4). The cryoprobes according to FIGS. 3 and 4 each include a probe head 15 which forms the distal end 11 of the cryoprobe 10. This probe head 15 is fastened to the probe shank 13. The probe head 15 and probe shank 13 surround a region which will be referred to hereinafter as the inner region of the probe 10. A gas supply means 20, which comprises a gas supply line 21 and a distal end 22 of the gas supply line 21, is located in this inner region. Gas is brought under high pressure into an expansion chamber 50 in the interior of the probe head 15 via the gas supply line. The gas issues into this expansion chamber 50 via an outlet opening and withdraws heat energy from the probe head 15 owing to the Joule-Thomson effect. The unfilled interior of the cryoprobe 10 forms a gas return means 40. That is to say, the issuing gas is removed in the interior of the probe 10 in the proximal direction, i.e. in the direction of the connection 70.

FIG. 5 shows the distal end 11 of a first example embodiment of the cryoprobe 10. This cryoprobe 10 is also composed of a shank 13 and a probe head 15 which are adhesively bonded to each other. The shank 13 and probe head 15 form an outer sheath of the cryoprobe 10. The gas supply line 21, which allows gas to flow under high pressure into the expansion chamber 50 via a nozzle opening 24 at the distal end 22 of the gas supply line 21, is located within the cryoprobe 10. The gas return means 40 includes openings 41 to 41″ produced by perforation of the outer sheath of the cryoprobe 10. The expanded gas can escape via these openings 41 to 41″ from the inner region of the cryoprobe 10, in particular from the expansion chamber 50, into the outer region of the cryoprobe 10. The openings 41 to 41″ are located close to the distal end 11 of the cryoprobe 10.

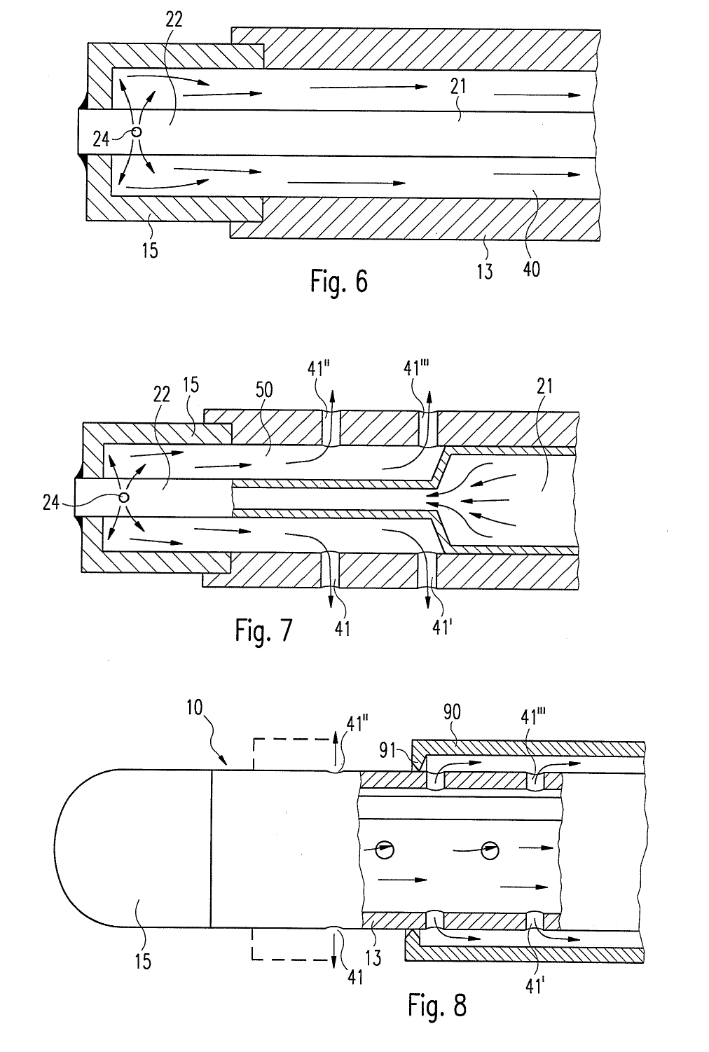

FIG. 6 shows a further cryoprobe 10, according to the prior art. The gas supply line 21, which is closed at its distal end 22 and forms a part of the probe head 15, extends along the longitudinal axis, in the inner region of the cryoprobe 10. A lateral nozzle opening 24, via which the gas flows into the interior of the cryoprobe 10, is located close to the distal end 22 of the gas supply line 21. The intermediate space between the gas supply line 21 and shank 13 of the cryoprobe 10 forms the gas return means 40.

FIG. 7 shows a second example embodiment of the cryoprobe 10. In its general construction, the cryoprobe 10 according to FIG. 7 is similar to that of FIG. 6. However, apart from a short portion close to the distal end 22 of the gas supply line 21, the gas supply line 21 takes up the entire inner region of the shank 13 of the cryoprobe 10. Close to the distal end 22, the gas supply line 21 tapers in such a way as to produce an intermediate space between the gas supply line 21 and shank 13 or probe head 15. This intermediate space serves as an expansion chamber 50 into which gas from the gas supply line 21 flows via the lateral nozzle opening 24. Furthermore, this intermediate space forms a part of the gas return means 40 which removes the expanded gas into the outer region of the probe via openings 41 to 41′″. Thus, a gas return means 40 in the proximal region of the shank 13 may be dispensed with.

FIG. 8 shows a third example embodiment of the cryoprobe 10. The construction of this cryoprobe 10 corresponds substantially to that of the cryoprobe 10 shown in FIG. 5. The gas return means 40 is formed partly by the interior of the cryoprobe 10 and also by the openings 41 to 41′″. The cryoprobe 10 is located within a protective tube 90 which can be used to remove the gas in a targeted manner in the proximal direction. A ring seal 91, which rests against the shank 13 of the cryoprobe 10 and distally seals the inner region of the protective tube 90 from the outer region, is located at the distal end of the protective tube 90. The protective tube 90 and the ring seal 91 are embodied so as to allow the cryoprobe 10 to be moved within the protective tube 90 in the distal and proximal directions. Depending on the position of the cryoprobe 10 within the protective tube 90 and depending on the position of the openings 41 to 41′″, the expanded gas is removed in its entirety or only in part via the inner region of the protective tube 90. In the position according to FIG. 8, the gas escapes directly into the organ via the openings 41, 41″, while the gas escaping through the openings 41′ and 41′″ penetrates the inner region of the protective tube 90 and is removed there in the proximal direction.

The probes according to the disclosed embodiments are able to have a much thinner diameter than those of the prior art, since all or a large percentage of the interior of the cryoprobes 10 can be filled by the gas supply line 21. As a result of the use of the protective tube 90, it is possible to use the probes according to the disclosed embodiments also in applications in which it is not desirable for the gas to escape directly into an organ or tissue.

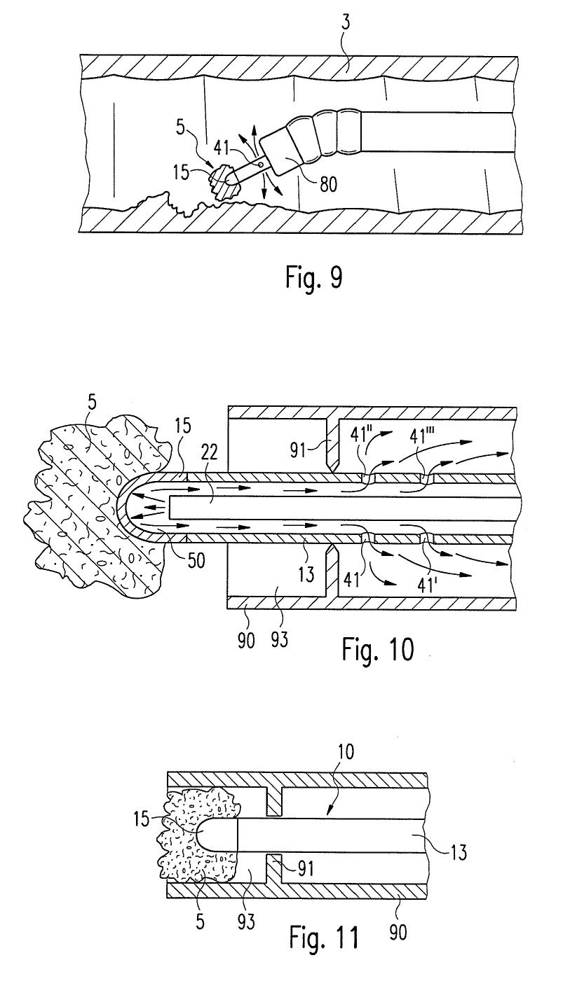

However, there are numerous fields of application in which it is harmless for small amounts of gas to escape. For example, FIG. 9 shows a cryoprobe 10 according to an example embodiment in the working channel of an endoscope 80 which is introduced into an intestinal tract. The gas necessary for freezing a tissue sample 5 solid escapes in this case directly into the intestinal tract via the opening 41. As a result, the patient is not placed in any danger.

FIGS. 10 and 11 show a development of the protective tube 90 from FIG. 8. As previously, the protective tube 90 with the ring seal 91 still serves at least partly to remove the gas issuing via the openings 41 to 41′″. However, the protective tube 90 of FIGS. 10 and 11 additionally includes a sample chamber 93 at the distal end of the protective tube 90.

The sample chamber 93 serves to receive a tissue sample 5. The cryoprobe 10 may be withdrawn into the interior of the protective tube 90 in such a way that the tissue sample 5 also comes to lie within the protective tube 90, namely in the sample chamber 93. This prevents the sample 5 from becoming stripped off when the cryoprobe 10 is extracted from the working channel of the endoscope 80.

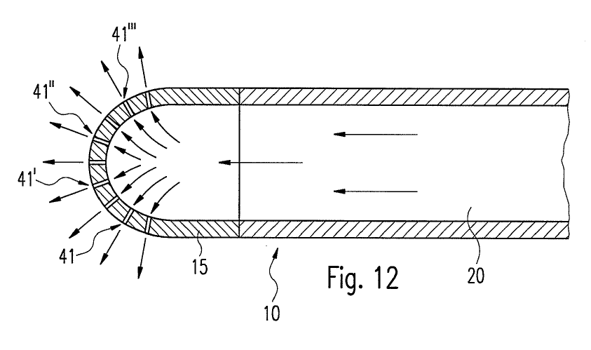

In the example embodiments of FIGS. 10 and 11, the sample chamber 93 is formed as a result of the fact that the ring seal 91 is not located directly at the distal end of the protective tube 90, but is slightly offset in the proximal direction. As a result, a portion of the inner region of the protective tube 90 is open in the distal direction for receiving the tissue sample 5. FIG. 12 shows a further example embodiment of the cryoprobe 10. The openings 41 to 41′″ are located directly at the distal end 11 of the cryoprobe 10. They are formed by a particle filter having a small hole diameter on the probe head 15. The openings 41 to 41′″ coincide with the region which receives the tissue sample 5. The entire interior of the cryoprobe 10 is used as the gas supply means 20. The exterior immediately before the probe head 15 serves in this example embodiment as the expansion chamber 50. The openings 41 to 41′″ thus have a double function. They are on the one hand the nozzle for the expansion of the gas and on the other hand part of the gas return means 40.

It should be noted at this point that all the aforementioned parts are claimed as essential to the invention both alone and in any combination, particularly the details shown in the drawings. Amendments thereof are the common practice of persons skilled in the art.

1-11. (canceled)

12. A cryosurgical instrument for obtaining a tissue sample with a probe, comprising:
the probe, including a probe head onto which the tissue sample is frozen and a probe shank for guiding the probe head to the tissue sample;
a supply means for supplying a fluid into an expansion chamber, the expansion chamber being configured such that the supplied fluid expands therein for cooling the probe head; and
a removal means connected to the expansion chamber for removing the expanded fluid, the removal means comprising at least one opening on a distal region of the probe which removes the expanded fluid into an outer region outside the probe.

13. The cryosurgical instrument according to claim 12, wherein the removal means comprises at least one opening at the distal end of the probe shank.

14. The cryosurgical instrument according to claim 12, wherein the removal means comprises at least one opening in the probe head.

15. The cryosurgical instrument according to claim 12, wherein the expansion chamber is arranged within the probe for cooling the probe head.

16. The cryosurgical instrument according to claim 12, further comprising an adapter for removing the expanded fluid in the proximal direction.

17. The cryosurgical instrument according to claim 16, wherein the adapter comprises a protective tube with a seal, the probe being movably mounted in the protective tube and the seal distally sealing an interior of the protective tube for receiving the expanded fluid from portions of the probe exterior to the protective tube.

18. The cryosurgical instrument according to claim 17, wherein the protective tube further comprises a region for receiving the tissue sample.

19. The cryosurgical instrument according to claim 18, wherein the region for receiving the tissue sample is located distally from the seal.

20. The cryosurgical instrument according to claim 17, wherein the adapter is adapted for introducing the expanded fluid into a working channel of an endoscope.

21. The cryosurgical instrument according to claim 12, wherein the supply means comprises a supply line arranged inside the probe shank.

22. The cryosurgical instrument according to claim 12, wherein the removal means comprises a particle filter on the probe head.

23. The cryosurgical instrument of claim 12, wherein the fluid is a gas.

24. A method for cooling a probe head of a cryosurgical probe, the method comprising:
supplying a fluid to an expansion chamber near the probe head;
expanding the fluid in such a way that heat energy is withdrawn from the probe head; and
removing the expanded fluid by diverting the expanded fluid from the interior of the probe in a distal region of the probe.

25. The method according to claim 24, wherein the fluid is supplied over a period of time which is less than or equal to five seconds.

26. The method of claim 24, wherein the fluid is a gas.
